(19) United States
(12) Patent Application Publication
(10) Pub. No.: US 2011/0285957 A1
(43) Pub. Date: Nov. 24, 2011

(54) Limited access and antifog protective eyewear for welding

(76) Inventors: William Mikulenka, El Campo, TX (US); Lawanda Mikulenka, El Campo, TX (US)

(21) Appl. No.: 12/800,838

(22) Filed: May 24, 2010

(51) Int. Cl. G02C 11/08; A61F 9/00

(52) U.S. Cl. 351/62; 2/15

(57) ABSTRACT

Items of protective eyewear and methods of use are disclosed, useable during welding, cutting, soldering, brazing, and other operations. The body of the eyewear includes a first side having a generally transparent surface, a second side for placement against a user's face, and space defined between the first and second sides for accommodating a second item of eyewear worn by the user if needed. A circulation apparatus secured to a first side surface of the eyewear is useable to evacuate air from the space to prevent fogging, while additional air is drawn through at least one ventilation opening is disposed through the body of the eyewear. A power source and a switch are secured to the body in operative association with the circulation apparatus, such that actuation of the switch causes actuation of the circulation apparatus.

### FIELD

The present disclosure relates, generally, to items of protective eyewear and methods of use, and specifically, to items of protective eyewear useable during welding, cutting, soldering, and/or brazing and methods for preventing fogging or other occlusion of such eyewear.

### BACKGROUND

When performing welding operations, cutting, soldering, and/or brazing, it is necessary to wear protective clothing, most notably, protective goggles and other eyewear to protect the wearer from harmful glares and other visual emissions, irritants, and debris. When welding, specifically, it is often necessary to wear a protective helmet and/or hood that covers much of a user's face, head, and/or neck, often over other eyewear, such as safety glasses. However, under many circumstances, such as when working within power plants and other tight quarters where space is restricted, commonly known as “limited access” circumstances, it is difficult, if not impossible, for a user to insert his or her head or face into a limited access location to visualize a weld, cut, or similar operation, while equipped with a conventional welding hood or helmet, or similar protective objects.

Welding and similar operations are further complicated by the hot, humid, and unfavorable conditions to which a user's eyewear is subjected. Due to such conditions, it is extremely common for the visor and/or filter plate of a user's hood, helmet, and/or goggles to become fogged, and for the user's safety glasses and/or prescription eyewear worn beneath the hood, helmet, and/or goggles to be similarly affected. Welders and other operations must cease operations frequently to clean safety glasses and/or prescription eyewear, as well as the visor and/or filter plate.

A need exists for protective eyewear useable for limited access welding, cutting, soldering, and/or brazing operations, while retaining a sufficient degree of protection.

A need also exists for protective eyewear having an integrated air circulation apparatus or similar device useable to prevent fogging or other types of occlusion of the front surface and any other safety or corrective glasses or other eyewear worn beneath.

Embodiments of the present invention meet these needs.

### BRIEF DESCRIPTION OF THE DRAWINGS

In the detailed description of various embodiments of the present invention presented below, reference is made to the accompanying drawings, in which:

FIG. 1 depicts a rear perspective view of an embodiment of an item of protective eyewear useable within the scope of the present disclosure.

FIG. 2 depicts a top perspective view of the eyewear of FIG. 1.

FIG. 3 depicts a right side perspective view of the eyewear of FIG. 1.

FIG. 4 depicts a left side perspective view of the eyewear of FIG. 1.

Embodiments of the present invention are described below with reference to the listed Figures.

### DETAILED DESCRIPTION OF THE EMBODIMENTS

Before explaining selected embodiments of the present invention in detail, it is to be understood that the present invention is not limited to the particular embodiments described herein and that the present invention can be practiced or carried out in various ways.

Embodiments of the present invention relate to an item of protective eyewear, useable during welding, cutting, soldering, brazing, and/or similar operations. The eyewear can generally include a first side, i.e. a front side, having an at least partially transparent surface, which can include a visor, lens, light filter, magnifier, or combinations thereof, and a second side, i.e. a back side, adapted to be placed against the face of a user. It should be understood that the front side of the eyewear can include one or more light filters and/or magnifying lenses removably secured thereto, pivotally or otherwise movably secured thereto, or combinations thereof, for enabling a user to selectively manipulate the eyewear to provide light filtration and/or magnification, as needed. One or more adjustable straps, belts, bands, ratcheting systems, or other adjustable fastening members, as known in the art, can be used to secure the eyewear about a user's head, such that the second side is retained against the user's face, to minimize the potential for debris and/or irritants to contact the user's eyes.

A space is defined between the first and second sides of the eyewear, the space being sized to accommodate a second item of eyewear, such as safety glasses and/or corrective glasses. Sizing of embodiments of the present eyewear to accommodate safety glasses and/or other additional items of eyewear can be of particular use during welding and other operations where use of safety glasses, in addition to a conventional hood or helmet, is required.

The body of the eyewear can, generally, include top, bottom, and side surfaces, with one or more ventilation openings disposed through the body. In an embodiment of the invention, the one or more ventilation openings can be provided through the top and/or bottom surfaces. An air circulation apparatus, such as fan, can be secured to a side surface of the body, oriented such that the air circulation apparatus will evacuate air from the space within the eyewear, while drawing additional air through the one or more ventilation openings. Use of an air circulation apparatus in this manner can prevent fogging or other occlusion of the front surface of the eyewear, as well as fogging and/or occlusion of any safety glasses or additional eyewear worn beneath. In an embodiment of the invention, one or more filtration members can be placed within the ventilation openings to prevent smoke, debris, or other irritants and/or contaminants from the air entering the eyewear.

A power source, such as one or more batteries, can be provided in electrical communication with the air circulation apparatus. In an embodiment of the invention, the power source can be secured to the body of the eyewear, such as to a side surface of the body, to minimize bulk and reduce the possibility of disconnection from the air circulation apparatus. A switch can also be secured to the body of the eyewear, in operative association with the power source and/or the air circulation apparatus, to enable actuation of the air circulation apparatus by a user.

In an embodiment of the invention, a protective plate, such as a sheet of Micarta®, or a similar electrically insulated composite and/or polymer, can be secured to the front of the eyewear, such as between the visor and/or other transparent surface(s) and the remainder of the body. The protective plate can be removeable and/or replaceable as needed. Additionally or alternatively, a hood formed from leather, fire-retardant cloth, or similar materials can be secured to the eyewear and moveable to at least partially cover a user's face, neck, and/or shoulders.

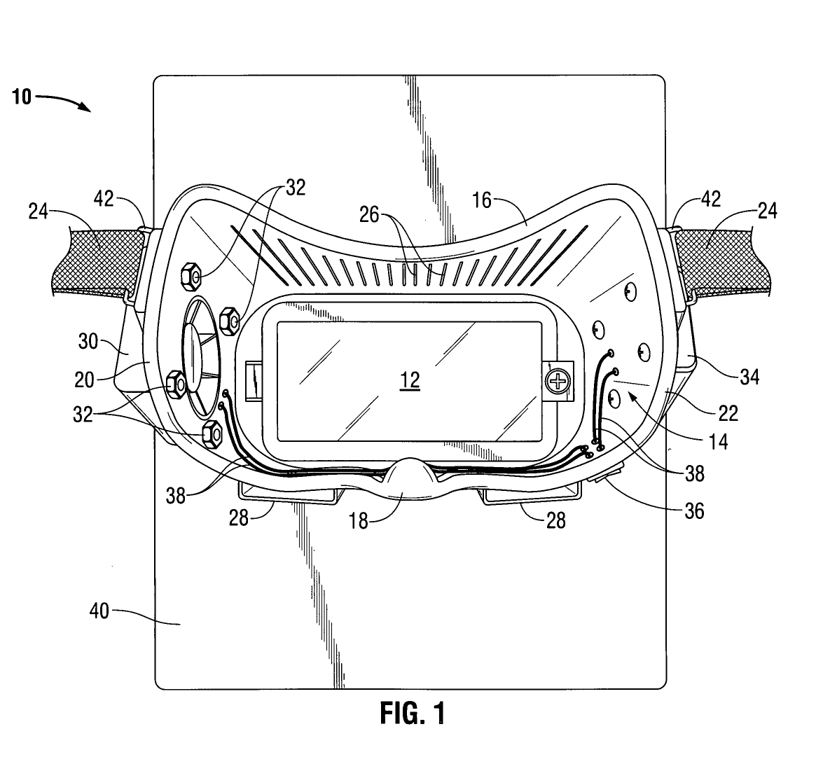

Referring now to FIG. 1, a rear view of an embodiment of protective eyewear (10) useable within the scope of the present disclosure is shown. The eyewear (10) includes a front side (12) having a generally transparent visor for viewing welding and other operations therethrough. The visor can be provided with moveable and/or removable lenses, light filters, and/or other coverings and/or modifiers, to facilitate adaptability for a variety of operations and conditions. The eyewear (10) also includes a back side (14) adapted for placement against a user's face such that the user's eyes are enclosed within the body of the eyewear (10). Specifically, the body of the eyewear (10) includes a top surface (16) a bottom surface (18), a first side surface (20), and a second side surface (22), such that when worn, the user's eyes are enclosed within the surfaces (16, 18, 20, 22) and the front side (12) to protect the user's eyes from irritants, debris, light, and other materials and/or emissions.

An adjustable strap (24) is shown secured to the side surfaces (20, 22) using two loops and/or buckles (42), for placement around a user's head to facilitate securing of the back side (14) of the eyewear (10) against the user's face. Other adjustable securing members, such as bands, belts, hook and loop fasteners, pin-based fasteners, and other such securing members and/or fasteners known in the art are also useable, in conjunction with any portion of the depicted eyewear (10).

The eyewear (10) is also shown having an upper ventilation opening (26) disposed through the top side (16) and two lower ventilation openings (28) disposed through the bottom side (18). It should be understood that any number and/or configuration of ventilation openings can be provided through any surfaces (16, 18, 20, 22) of the eyewear (10). Additionally, in an embodiment of the invention, one or more filtering members can be disposed through any of the ventilation openings (26, 28) to prevent contaminants and/or impurities from passing through the ventilation openings (26, 28) and entering the eyewear (10).

A fan (30) is shown secured to the first side surface (20) of the eyewear (10), using a plurality of fasteners (32), which are depicted as screws threaded through the first side surface (20), and secured using nuts. It should be understood that while the fan (30) is depicted secured to the first side surface (20) using #8-36×0.75″ screws and corresponding nuts, any type of air circulation apparatus can be secured to any portion of the eyewear (10), using any manner of fasteners, adhesives, or other means. While the size and type of fan (30) or other air circulation apparatus used can vary, FIG. 1 depicts the fan (30) as a 12-volt, DC-operable fan, having an approximate length of 50 millimeters, an approximate width of 50 millimeters, and an approximate thickness of 15 millimeters, capable of rotating at 5200 revolutions per minute and flowing air at a rate of 15 cubic feet per minute.

FIG. 1 further depicts a power source (34) secured to the second side surface (22). Specifically, the power source (34) is shown as a 9-volt battery within a holder, secured to the eyewear (10) using a plurality of aluminum rivets or similar fasteners. It should be understood that any manner of power source (34) can be secured to any portion of the eyewear (10) in any manner, depending on the requirements of the fan (30) or other air circulation apparatus used. A manual switch (36) is further depicted secured to the bottom surface (18) of the eyewear (10). Wiring (38) or other electrically conductive members can be used to place the fan (30), power source (34) and switch (36) into electrical communication. As such, the switch (36) is operable by a user to complete a circuit, thereby causing power from the power source (34) to be conducted to the fan (30). Actuation of the fan (30) evacuates air or another gas from the interior of the eyewear (10), while drawing additional air into the eyewear (10) through the ventilation openings (26, 28).

FIG. 1 also depicts a protective faceplate (40) secured to the front surface (12) of the eyewear (10), for providing protection to a user's face during welding or similar operations. While the dimensions and material of the faceplate (40) can vary, the depicted faceplate (40) is shown as a piece of Micarta™ having a width of about 8 inches, a height of about 9 inches, and a thickness of about 0.0625 inches. A slot having a width of about 4.525 inches and a height of about 0.3125 inches is provided through the faceplate (40) for accommodating the front surface (12) of the eyewear (10). For example, the faceplate (40) can be installed between a lens, visor, and/or light filter and the body of the eyewear (10). In an embodiment of the invention, a fire-retardant and/or protective hood can also be secured to the eyewear (10) and moveable to cover the face, neck, and/or shoulders of a user.

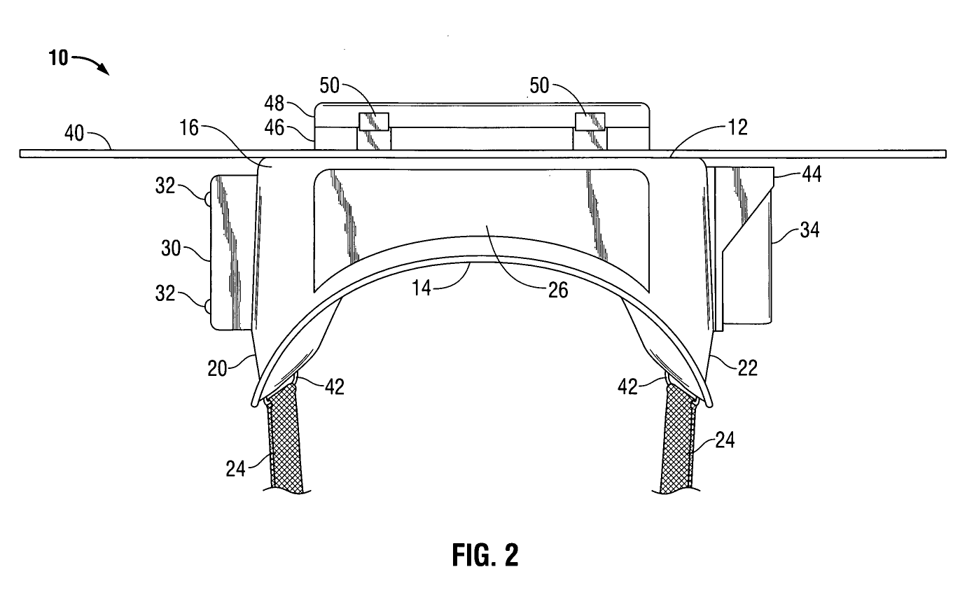

Referring now to FIG. 2, a top view of the eyewear (10) if FIG. 1 is depicted, showing the front side (12) displaced from the back side (14) to define a space therebetween sufficient to accommodate safety and/or corrective glasses, or another second item of eyewear if needed. The top surface (16) and side surfaces (20, 22) of the eyewear (10) are also shown, the top surface (16) including the upper ventilation opening (26), as described previously. While FIG. 2 depicts a single, generally elongate series of ventilation openings, it should be understood that any number and configuration of continuous or discontinuous ventilation openings can be disposed through any portion of the body of the eyewear (10). The adjustable strap (24) is also shown secured at each end to the side surfaces (20, 22) of the eyewear (10) using two loops and/or buckles (42); however, it should be understood that any securing members or other forms of attachment known in the art can be used.

The fan (30) is shown secured to the first side surface (20) using fasteners (32), while the power source (34) is shown secured to the second side surface (22) within a holder (44) adapted to conduct power from the power source (34) to the fan (30). The protective faceplate (40) is also show secured to the front side (12) of the eyewear (10), between the body of the eyewear (10) and a lens and/or visor (46). A light filter attachment (48) is shown pivotally attached to the lens and/or visor (46) using hinges (50) to enable a user to selectively manipulate the light filter attachment (48) during welding or other operations.

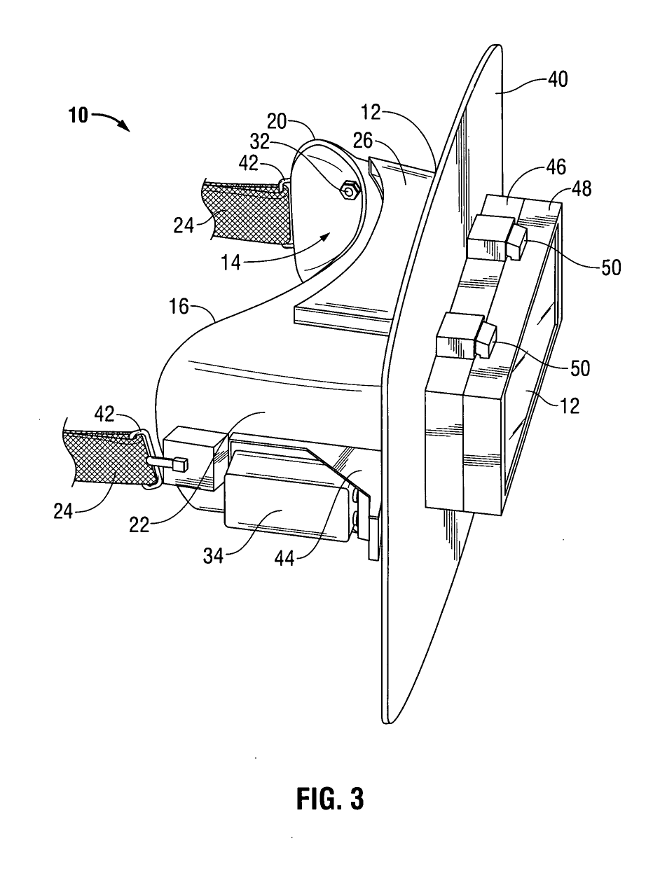

Referring now to FIG. 3, a right side view of the eyewear (10) of FIG. 1 is shown, which depicts the front side (12) displaced from the back side (14) to define a space therebetween, as described previously. The power source (34) is shown secured within the holder (44) disposed on the second side surface (22), opposite the fan (not visible in FIG. 3) secured to the first side surface (20) using one or more fasteners (32). The adjustable strap (24) is also shown secured to the side surfaces (20, 22) using buckles (42).

FIG. 3 also shows the protective faceplate (40) removably secured to the front side (12) of the eyewear (10), between the body of the eyewear (10) and the lens and/or visor (46), with the light filter attachment (48) pivotally attached thereto using hinges (50). Use of a manually moveable light filter attachment (48) enables a user to selectively view a weld or similar operation. In an embodiment of the invention, the light filter attachment (48) and lens and/or visor (46) can be removed to enable removal and/or replacement of the protective faceplate (40).

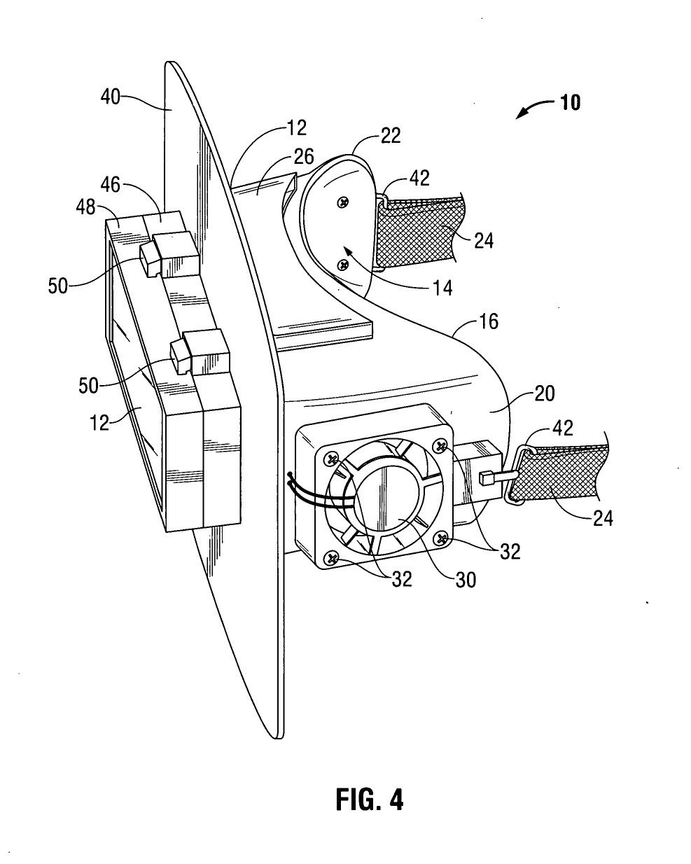

Referring now to FIG. 4, a left side view of the eyewear (10) of FIG. 1 is depicted. The fan (30) is shown secured to the first side surface (20) using fasteners (32). The adjustable strap (24) is also shown secured to the side surfaces (20, 22) using buckles (42). The protective faceplate (40) is shown secured between the body of the eyewear (10) and the lens and/or visor (46), which is depicted having a light filter attachment (48) pivotally engaged therewith via hinges (50), as described previously.

The depicted eyewear (10) thereby provides sufficient protection for use during welding, cutting, soldering, brazing, and similar operations, while having dimensions sufficiently small to enable use during “limited access” operations. Additionally, use of the fan (30) or other air circulation apparatus, in conjunction with the ventilation openings (26, 28) prevents fogging and/or other types of occlusion during use, providing enhanced efficiency and safety.

While various embodiments of the present invention have been described with emphasis, it should be understood that within the scope of the appended claims, the present invention might be practiced other than as specifically described herein.

What is claimed is:

1. An item of protective eyewear for use during welding, cutting, soldering, brazing, or combinations thereof, the eyewear comprising:
a body having a first side comprising an at least partially transparent surface, a second side adapted for placement against a face of a user, a top surface, a bottom surface, a first side surface, and a second side surface, wherein a space is defined between the first side and the second side;
at least one ventilation opening disposed through the body;
a circulation apparatus secured to the body, wherein the circulation apparatus is oriented to evacuate air from the space to prevent fogging of the at least partially transparent surface, and wherein additional air enters the space through said at least one ventilation opening;
a power source secured to the body in operative association with the circulation apparatus; and
a switch secured to the body in operative association with the power source, the circulation apparatus, or combinations thereof, wherein actuation of the switch causes actuation of the circulation apparatus.

2. The eyewear of claim 1, further comprising a protective plate secured to the body, wherein the protective plate at least partially covers the face of the user.

3. The eyewear of claim 2, wherein the protective plate is secured between the at least partially transparent surface and the body.

4. The eyewear of claim 1, further comprising a protective hood disposed around the body, wherein the protective hood is moveable to at least partially cover the face of the user.

5. The eyewear of claim 1, wherein said at least one ventilation opening is disposed through the top surface of the body, the bottom surface of the body, or combinations thereof.

6. The eyewear of claim 1, wherein the circulation apparatus comprises a fan.

7. The eyewear of claim 1, wherein the power source is secured to the second side surface.

8. The eyewear of claim 1, further comprising a filtering member disposed within said at least one ventilation opening for removing impurities from air that passes through said at least one ventilation opening.

9. The eyewear of claim 1, wherein the second item of protective eyewear comprises safety glasses, corrective glasses, or combinations thereof.

10. The eyewear of claim 1, further comprising a magnifying lens, a light filter, or combinations thereof removably secured in an at least partially overlapping relationship with the at least partially transparent surface.

11. The eyewear of claim 1, further comprising a magnifying lens, a light filter, or combinations thereof movably secured to the at least partially transparent surface.

12. The eyewear of claim 1, wherein the space between the first side and the second side is sized for accommodating a second item of eyewear worn by the user.

13. A method for preventing occlusion of eyewear during welding, cutting, soldering, brazing, or combinations thereof, the method comprising:
providing an item of protective eyewear over a face of a user, wherein the item of protective eyewear comprises a circulation apparatus disposed in a surface and at least one ventilation opening disposed through an additional surface, and wherein the item of protective eyewear defines a space; and
actuating the circulation apparatus to evacuate air from the space of protective eyewear, wherein actuating of the circulation apparatus transfers air from the space to an exterior of the item of protective eyewear, and wherein additional air enters the space through said at least one ventilation opening.

14. The method of claim 13, wherein the step of providing the item of protective eyewear over the face of the user comprises at least partially covering the face of the user with a protective plate, a protective hood, or combinations thereof secured to the item of protective eyewear.

15. The method of claim 13, wherein the step of actuating the circulation apparatus draws the additional air through said at least one ventilation opening disposed through a surface of the item of protective eyewear.

16. The method of claim 13, wherein the step of actuating the circulation apparatus comprises providing power to the circulation apparatus from a power source secured to the item of protective eyewear.

17. The method of claim 13, wherein the step of actuating the circulation apparatus comprises actuating a switch in operative communication with the circulation apparatus.

18. The method of claim 13, wherein the step of actuating the circulation apparatus further comprises drawing the additional air through a filtering member disposed within said at least one ventilation opening to remove impurities from the additional air.

19. The method of claim 13, wherein the step of providing the item of protective eyewear over the face of the user comprises providing the item of protective eyewear over a second item of protective eyewear worn by the user such that the second item of protective eyewear is disposed within the space.

20. An item of protective eyewear comprising:
a body having a first side comprising an at least partially transparent surface, a second side adapted for placement against a face of a user, and at least one side surface, wherein a space is defined between the first side and the second side;
at least one ventilation opening disposed through the body; and
a circulation apparatus secured to said at least one side surface, wherein the circulation apparatus is oriented to evacuate air from the space to prevent fogging of the at least partially transparent surface, and wherein additional air enters the space through said at least one ventilation opening.
